(19) United States
(12) Patent Application Publication
(10) Pub. No.: US 2004/0248106 A1
(43) Pub. Date: Dec. 9, 2004

(54) Clinical array assays that include a sample quality evaluation step and compositions for use in practicing the same

(76) Inventors: Leslie A. Leonard, Portola Valley, CA (US); Carol T. Schembri, San Mateo, CA (US); Laurakay Bruhn, Mountain View, CA (US); Michael T. Barrett, Mountain View, CA (US); Paul K. Wolber, Los Altos, CA (US); Richard J. Pittaro, San Carlos, CA (US); Douglas A. Amorese, Los Altos, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/456,874

(22) Filed: Jun. 6, 2003

(51) Int. Cl. C12Q 1/68

(52) U.S. Cl. 435/6

(57) ABSTRACT

Array-based clinical assays and compositions for use in practicing the same are provided. A feature of the subject array-based clinical assays is that they include a sample quality evaluation step that is independent from the clinical assay step of the assays, where the sample quality evaluation step may be performed in a number of different ways. Also provided are compositions, devices and kits for use in practicing the subject methods.

### INTRODUCTION

1. Field of the Invention

The present invention relates to biopolymeric arrays, particular as employed in clinical assay applications, e.g., expression based clinical assays.

2. Background of the Invention

Array assays between surface bound binding agents or probes and target molecules in solution may be used to detect the presence of particular biopolymeric analytes in the solution. The surface-bound probes may be oligonucleotides, peptides, polypeptides, proteins, antibodies or other molecules capable of binding with target biomolecules in the solution.

One typical array assay method involves biopolymeric probes immobilized in an array on a substrate such as a glass substrate or the like. A solution containing target molecules (“targets”) that bind with the attached probes is placed in contact with the bound probes under conditions sufficient to promote binding of targets in the solution to the complementary probes on the substrate to form a binding complex that is bound to the surface of the substrate. The pattern of binding by target molecules to probe features or spots on the substrate produces a pattern, i.e., a binding complex pattern, on the surface of the substrate that is detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

Such array assays find use in a variety of different fields, e.g., genomics (in sequencing by hybridization, SNP detection, differential gene expression analysis, identification of novel genes, gene mapping, finger printing, etc.) and proteomics.

One particular area in which such array-based assays are finding increasing use is in clinical assays, e.g., in which the array assays are performed in a clinical setting to diagnose and/or monitor the progression of a condition in a patient, e.g., a disease condition.

For array-based assays performed in the clinical setting, e.g., expression-based clinical assays, a given sample is typically obtained at a location remote from the assay location, e.g., collected in a clinic elsewhere in a hospital, and then transported to a central laboratory for clinical assay. For example, the sample may be collected at an independent clinic and forwarded to a reference lab, etc., for expression-based testing to obtain a clinical result. In these cases, the sample may be exposed to variations in delivery-time, temperature and mixing, etc. Alternatively, a sample may be inadvertently collected in the wrong container, or may be mislabeled. The professional performing the clinical array-based assay usually has little way of knowing the quality of the sample until the test is run. In most situations, if a given set of results falls outside of an established set of parameters, the results are “flagged” to indicate that the results cannot be reported with confidence. As such, in current practice the quality of the sample is assessed, if at all, only from the clinical assay results per se, in the sense that if the assay results do not meet predetermined criteria, the sample quality is viewed as suspect.

Because sample quality can have a significant impact on the value of results obtained in a given clinical assay, it would be desirable to have protocol that could, for example, include a way to ensure that a given sample has been appropriately collected, processed, labeled, transported and/or stored prior to being clinically assayed, where this quality assurance would be provided by an assay sub-step that was independent of the clinical assay portion of the protocol. Such an independent quality assurance sub-step could, in one or more embodiments, offer one or more advantages, for example a more reliable determination of sample and therefore results quality, cost savings in that a sample may not be clinically assayed if it does not meat a threshold quality, and the like. The present invention satisfies this need.

Relevant Literature

Representative references that disclose array-based clinical assays include: WO 02/056030; WO 02/084249; WO 02/33415; WO 02/39120; U.S. Pat. Nos. 6,210,878 and 6,171,793.

### SUMMARY OF THE INVENTION

Array-based clinical assays and compositions for use in practicing the same are provided. A feature of the subject array-based clinical assays is that they include a sample quality evaluation step that is independent from the clinical assay step of the assays, where the sample quality evaluation step may be performed in a number of different ways. Also provided are compositions, devices and kits for use in practicing the subject methods.

### BRIEF DESCRIPTION OF THE DRAWINGS

FIG. 1A provides a depiction of a first embodiment of a sample containment device according to one embodiment of the subject invention.

FIG. 1B provides a depiction of a sample containment device according to a second embodiment of the subject invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer of one or more types of repeating units. Biopolymers are typically found in biological systems and particularly include polysaccharides (such as carbohydrates), peptides (which term is used to include polypeptides and proteins) and nucleic acids, as well as their analogs such as those compounds composed of or containing amino acid analogs or non-amino acid groups, or nucleotide analogs or non-nucleotide groups.

A “biomonomer” references a single unit, which can be linked with the same or other biomonomers to form a biopolymer (e.g., a single amino acid or nucleotide with two linking groups one or both of which may have removable protecting groups).

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids which are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The term “array” encompasses the term “microarray” and refers to an ordered array presented for binding to nucleic acids and the like.

An “array,” includes any one, two-dimensional or substantially two-dimensional (as well as a three-dimensional) arrangement of addressable regions bearing biopolymers, e.g., nucleic acids, polypeptides, and the like. Where the arrays are arrays of nucleic acids, the nucleic acids may be adsorbed, physisorbed, chemisorbed, photo-induced cross-linked, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100 μ², or even smaller. For example, features may have widths (that is, diameter, for a round spot) in the range from a 1 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Array substrates may be flexible (such as a flexible web). When the substrates are flexible, theymay be of various lengths including at least 1 m, at least 2 m, or at least 5 m (or even at least 10 m). “Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having alength greater than a width. For example, the web length to width ratio may be at least 5/1, 10/1, 50/1, 100/1, 200/1, or 500/1, or even at least 1000/1.

With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm. Array substrates may also be reflective and have little or no transparency. The reflectivity may reduce the absorption of the incident illuminating laser light and subsequent heating if the focused laser beam travels too slowly over a region. The substrate may be at least 20% reflective, preferably at least 50% reflective.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular probe sequence. Array features are typically, but need not be, separated by intervening spaces. In the case of an array in the context of the present application in certain embodiments, the “target” will be referenced as a moiety in a mobile phase (typically fluid), to be detected by “probe” which is bound to the substrate at the various regions. However, in certain embodiments, e.g., Comparative Genomic Hybridization embodiments (CGH), it may be more appropriate to refer to the substrate surface immobilized entities as targets and the fluid phase analytes as probes.

A “scan region” refers to a contiguous (for example, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

By “remote location,” it is meant a location other than the location at which the array is present and hybridization occurs. For example, a remote location could be another location (e.g., office, lab, etc.) in the same building, city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electronic signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, words such as “top,” “upper,” and “lower” are used in a relative sense only.

The term “stringent assay conditions” as used herein refers to conditions that are compatible to produce binding pairs of probes and targets of sufficient complementarity to provide for the desired level of specificity in the assay while being incompatible to the formation of binding pairs between binding members of insufficient complementary to provide for the desired specificity. An example of stringent assay conditions is rotating hybridization at 65° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based systemh are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DETAILED DESCRIPTION OF THE INVENTION

Array-based clinical assays and compositions for use in practicing the same are provided. A feature of the subject array-based clinical assays is that they include a sample quality evaluation step that is independent from the clinical assay step of the assays, where the sample quality evaluation step may be performed in a number of different ways. Also provided are compositions, devices and kits for use in practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

In further describing the invention in greater detail than provided in the Summary and as informed by the Background and Definitions provided above, representative embodiments of the subject array-based clinical assays are described first in greater detail, followed by a discussion of representative devices, systems and kits that find use in the subject methods.

Methods

As summarized above, the subject methods are array-based clinical assay methods. By “array-based” is meant that the assay protocols of the subject invention employ an array (as defined above) to assay or test a given sample. As such, in the subject array-based assays, a sample is contacted with an array and binding complexes on the surface of the array are then detected to provide an assay result.

By “clinical assay” is meant an assay or test that is performed on a sample obtained from a host or subject in order to provide information on current health or condition, diagnosis, prognosis, treatment, prevention, and/or monitoring of a condition of the host or subject. The host or subject from which the sample is obtained may be a variety of different organisms, but is generally an animal, where animals of interest in many embodiments are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In many embodiments, the hosts or subjects from which the sample is obtained in the subject methods will be humans.

The sample may be any of a variety of different physiological samples that are obtainable from a host or subject, where representative samples of interest include, but are not limited to: whole blood, plasma, serum, semen, saliva, tears, urine, fecal material, sweat, buccal fluid, skin fluid, spinal fluid and hair; in vitro cell cultures, including a growth medium, cells and cell components; tissue biopsies and samples, surgically-excised tissues, and the like The sample may or may not be pretreated, e.g., by the addition of one or more agents of interest, such as preservatives, chaotropic agents, labeling agents, etc., as is known in the art.

As mentioned above, the assays are clinical assays, which means that the assays are conducted to provide information on current health or condition, diagnosis, treatment, prevention, and/or monitoring of a condition of the host or subject of interest. In other words, the assays are conducted to detect the presence of and/or determine the stage of, severity of, etc., a condition of the host. The condition may or may not be a disease condition. As such, in certain embodiments, the clinical assay is performed to diagnose the presence of, and/or determine the stage or monitor the progression of, a disease condition. In yet other embodiments, the condition may not be a disease condition, but merely a propensity or predisposition for a disease condition. In yet other embodiments, the condition may not be a classical disease condition, but merely a physiological state that can be detected and/or monitored by array based assay, e.g., a metabolic rate determination, etc.

The nature of the array-based assays may vary, where the assays may be: genomic assays, in which nucleic acid targets in the sample are hybridized to an array of nucleic acid probes on the array; proteomic assays, in which protein analytes in the sample are specifically bound to an array of proteinaceous binding agent probes on the array; or a combination of a nucleic acid and protein array in which one or the other is the binding agent and the other is then used in the detection of the analyte; or other types of array assays using other types of arrays, usually biopolymeric arrays, to detect the presence of one or more analytes of interest in the sample. Arrays of interest include those described above.

In general, assay protocols performed according to the subject methods include the following three steps: (1) sample obtainment; (2) sample storage; and (3) assay of the sample. In the first sample obtainment step, a sufficient amount of sample is obtained from the host or subject of interest. In many embodiments, the sample is a fluid sample, where the volume of sample obtained in this step may range from, in fluid volumes, from about a few pL(equaling one or several cells) to about 10 mL (as in a blood sample from a human) to much larger quantities such as blood or urine samples from horse or other large animals, and in the case of tissue samples, from about 1-10 cells(or pgs tissue) to about 10^{6 or} 10⁷ cells (ng μgs tissue) in certain embodiments. The sample is typically obtained and placed in a sample containment means, in which it is then stored for a period of time in the second sample storage step of the subject protocols. The period of time during which the sample is stored in this step may vary, where the sample is stored typically for at least about several minutes to about 30 minutes or more, but may frequently be overnight such as at least about a week, where the period of time during which the sample is stored may be as long as a year or longer, such as years, decades or longer, where in certain embodiments the sample may be transported or moved from a first location to a second location. In the third step of the subject array-based clinical assay protocols, the sample is assayed using an array, as described above.

A feature of the subject clinical array-based assays is that they include a sample quality evaluation step, where the sample quality evaluation step is distinct from the clinical assay step. In other words, the sample quality evaluation step is a separate step of the assay protocol from the clinical assay step, such that the sample quality evaluation step is performed regardless of whether the clinical assay step is performed. In other words, the clinical assay result of the clinical assay step is not employed in the quality evaluation step. Viewed another way, a sample quality signature or profile that is separate from any clinical assay result is obtained for a sample being assayed according to the subject invention. Accordingly, while the quality evaluation step and the clinical assay step may be performed simultaneously or sequentially in the overall assay protocol (e.g., the same nucleic acid array may be employed in both the quality evaluation step and the clinical assay step, where both steps are performed at the same time), the quality evaluation step does necessarily not depend on completion of the clinical assay step, and is performed regardless of whether the clinical assay step is or is not performed In this manner, the subject methods are distinguished from prior art methods in which the results of a clinical assay are flagged if they do not satisfy a predetermined set of criteria, and the quality of the sample employed in the assay is indirectly evaluated from the clinical assay results.

The sample quality evaluation step of the subject clinical assay protocols or methods may be performed in any convenient manner that provides an independent evaluation of the sample quality to be assayed, which determination is not based on the clinical assay results themselves. A number of representative sample quality evaluation approaches are described below, where in certain representative embodiments, the sample quality is evaluated without the use of one or more quality indicators added to the sample, while in other embodiments the sample quality is evaluated by using one or more different quality indicator elements that are added to the sample upon obtainment of the sample from the host. Each of these types of representative embodiments will now be described separately in greater detail below.

### EMBODIMENTS WHERE ADDED QUALITY INDICATORS ARE NOT EMPLOYED

In certain embodiments, a sample quality evaluation protocol is employed that does not require the use of an added quality indicator to the sample upon obtainment of the sample from the source host/subject. As such, quality evaluation protocols of these embodiments do not include a step of adding a quality indicator to the sample, where the quality indicator is later employed in the quality evaluation protocol.

In certain embodiments of this type, the sample may be screened at the time of clinical assay for the presence of a quality indicative analyte, where the quality indicative analyte may be a variety of different types of analytes, so long as the detection thereof provides information about the quality of the sample at the time of clinical assay, e.g., its detection provides a quality signature of the sample.

In certain of these embodiments, the quality indicative analyte may be an analyte that is a contaminant, where detection of the contaminant indicates that the quality of sample has been compromised by the time the sample is employed in the clinical assay. Contaminants of interest include a variety of different types of molecules, including but not limited to: nucleic acids, polypeptides, polysaccharides, small organic molecules, metabolites, inorganic molecules, and the like. In certain embodiments of interest, the quality indicative analyte is one or more nucleic acid contaminants. By nucleic acid contaminant is meant a nucleic acid whose presence and/or amount in the sample is indicative of contamination of the sample, and therefore compromise of the sample, at the time of sample clinical assay. In these embodiments, the nucleic acid quality indicative analyte that is detected in the quality evaluation step may be nucleic acids that are present in the sample because of contaminating tissues in the sample, nucleic acids that are present in the sample in amounts that result from the presence of contaminating tissues/cells in the sample, nucleic acids present in the sample because of the presence in the sample of a contaminating biological source, such as bacterial contaminants, viral contaminants, organismal contaminants,etc. Tissue contaminants may indicate an undesirable, heterogeneous sample. For example, in the case of tumor biopsy or surgical extraction, the sample may contain both tumor cells and non-tumor cells, such as epithelial, stromal, immune-derived cells, etc. In the case of organismal contamination, an animal sample may or may not be expected to contain bacterial, viral nucleic acids, or other foreign contaminants, of which there are many examples, Hemophilus influenza, hepatitis, Cytomegalovirus, HIV, CMV, protozoal organisms, and/or prions Alternatively, probes on the nucleic acid array used for assessment may sample for cell or chromosome specific nucleic acids to confirm that the appropriate tissue sample is present.

Where the quality indicative analyte is made up of one or more nucleic acid analytes, as discussed above, the nucleic acid quality indicative element may be detected (and where desired quantitated) using any convenient nucleic acid detection protocol. In certain embodiments of interest, the nucleic acid detection protocol that is employed is an array-based nucleic acid detection protocol, where the sample is contacted with an array of probe nucleic acids that are specific for the one or more nucleic acids of the nucleic acid quality indicative element. This array may or may not be the array that is employed in the clinical assay step, where the use of the array in the quality evaluation step may or may not occur at the same time as the clinical assay. Upon contact and subsequent detection of surface bound duplexes, the resultant duplexes are employed to determine the presence of the nucleic acid quality indicative element in the sample and whether or not the sample has been contaminated.

In certain embodiments, a device that records or keeps track of at least one physical parameter or characteristic of the sample-is employed to evaluate the quality of the sample. The device in these embodiments is one that records a physical characteristic of the sample at least once during the time between its obtainment and the quality evaluation time, which preferably occurs at a time that is at least substantially the same time, if not the same time, as the clinical assay step. By physical characteristic/parameter is meant at least one physical feature of the sample, where physical features of interest include, but are not limited to: temperature, exposure to air/outside environment, time between sample obtainment and clinical assay, and the like. The device could be employed to observe a single type of physical characteristic, or a plurality of different physical characteristics. Where more than one physical parameter or physical characteristic is measured, the number of different characteristics that are measured may range from 2 to about 20, including 2 to about 10, etc.

A given physical parameter or characteristic may be measured a single time during the period between sample obtainment and quality evaluation, or a number of times, including continuously, during this storage period, such that the sample may be monitored during this storage period for the one or more physical parameter/characteristic of interest. Alternatively, it may record extremes, such as the highest temperature exposure or exposure beyond a specified parameter such as temperature or time.

The device employed in these embodiments may be any convenient device that is capable of adequately measuring the parameter(s) of interest in the sample at the appropriate time. In certain embodiments, the device employed is a sample containment element (which may be a stand alone element, e.g., as shown in the figures, or a component of an array-based assay integrated system, such as a separate compartment of an array chamber device), with a sensor element that determines or measures the physical parameter(s) of interest in the sample, when the sample is placed into the containment means upon obtainment. In other words, the device is a sample holder into which the sample is placed upon obtainment that includes a built-in or integrated sensor element(s) that detects or measures the physical parameter data of interest prior to the quality evaluation step.

The sample containment device may have any convenient structure that provides for the ability to hold a volume or quantity of a fluid sample. In certain embodiments, the containment device includes a “test-tube” like structure for holding a quantity of fluid. Such structures are well known in the clinical testing art. In certain embodiments, the volume of the containment element ranges from about 100 μl (capillary tubes) to about 50 ml, such as from about 1 ml to about 5 ml.

In certain embodiments of interest, the sensor element of the containment device includes a triggering element that is responsive to placement of the sample in the containment element, such that the sensor is activated upon addition of the sample to the containment element. A variety of triggering or actuating elements may be employed, where representative elements of interest include, but are not limited to: a fluid responsive element, e.g., that is actuated upon wetting by the sample; a temperature response element, e.g., that is actuated upon a change in temperature caused by placement of the sample in the containment element; a physical responsive element, e.g., a trigger that actuated upon piercing of a septum by a needle; a trigger that is actuated upon connection to the containment element, e.g., a trigger on a security cap that is actuated when the security cap is placed on the containment element; etc.

The sensor element may be a variety of different types of sensors, depending the physical parameter or parameters of interest. For example, representative sensor elements of interest include, but are not limited to: temperature sensors, e.g., present on the inside of the containment element, on a needle or other object that extends into the sample, on a security cap that is placed on the sample holder following obtainment, etc; air/external environment exposure sensors, e.g., an electronic seal that is capable of detecting a break in the seal, etc.; light exposure, e.g., photoesensitive sensor devices that detect exposure of the sample to light; degradation sensors, e.g., fiber optic elements with a fixed gap that allow multiple UV spectra of the solution to be taken over time without opening the vial; etc.

The sample containment devices also may include a data-recording element that records datum inputs from the sensor elements and stores them for use in the subsequent quality evaluation step. The data -recording element may be any of a variety of different elements, such as a silicon chip that stores the obtained physical information in volatile memory and from which the stored information can be retrieved during the sample quality evaluation step.

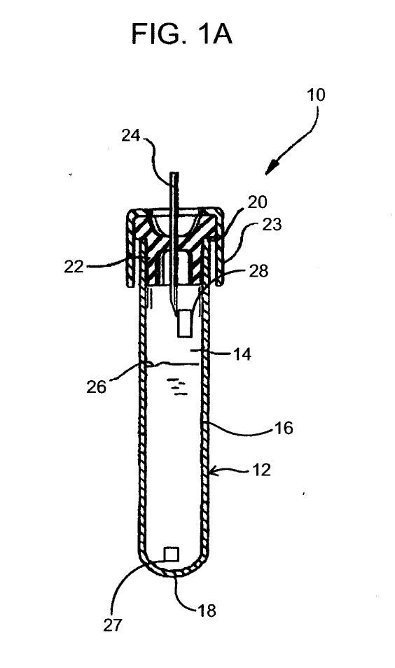

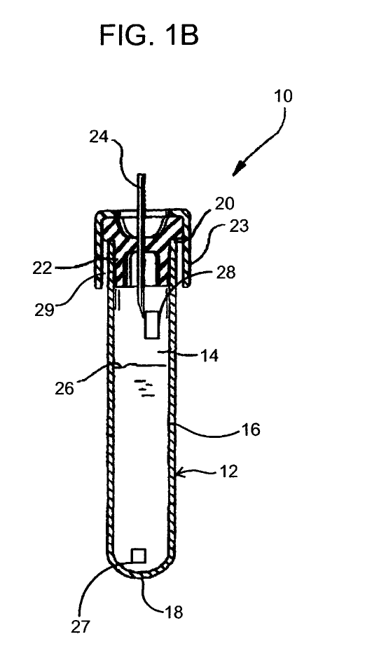

For purposes of further illustration only, two representative blood sample fluid containment means that include physical monitoring components are now further described with references to FIGS. 1A and 1B.

In one embodiment shown in FIG. 1A, device 10 is a device for drawing a blood sample directly from an animal, and particularly a human patient. Referring to FIG. 1A, device 10 includes a container 12 defining a chamber 14. In the embodiment illustrated, container 12 is a hollow tube having a side-wall 16, a closed bottom end 18 and an open top end 20. Container 12 is dimensioned for collecting a suitable volume of a biological fluid, e.g., blood. A resilient closure 22 is positioned in open top end 20 to close container 12. Preferably, closure 22 forms a seal capable of effectively closing container 12 and retaining a biological sample in chamber 14. A protective shield 23 overlies closure 22.

Container 12 can be made of glass, plastic or other suitable materials. Plastic materials can be oxygen impermeable materials or contain an oxygen impermeable layer. Alternatively, container 12 can be made of a water and air permeable plastic material.

In certain embodiments of interest, chamber 14 maintains a pressure differential between atmospheric pressure and is at a pressure less than atmospheric pressure. The pressure in chamber 14 is selected to draw a predetermined volume of a biological sample into chamber 14. Typically, a biological sample is drawn into chamber 14 by piercing closure 22 with a needle 24 or cannula as known in the art. An example of a suitable container 12 and closure 22 are disclosed in U.S. Pat. No. 5,860,937, the disclosure of which is hereby incorporated by reference in its entirety.

In many embodiments, the container 12 is fabricated from glass or a suitable transparent thermoplastic material, where representative materials of interest include, but are not limited to: polycarbonates, polyethylene, polypropylene, polyethylene-terephthalate, etc. Container 12 has a suitable dimension selected according to the required volume of the biological sample being collected. In one embodiment, container 12 has a tubular shape with an axial length of about 100-mm and a diameter of about 13-mm to 16-mm.

Closure 22 is made of a resilient material capable of maintaining an internal pressure differential less than atmospheric and that can be pierced by a needle or other cannula to introduce a biological sample into container 12. Suitable materials for closure include, for example, silicone rubber, natural rubber, styrene butadiene rubber, ethylene-propylene copolymers and polychloroprene.

A feature of the embodiment shown in FIG. 1A is sensor 26, which monitors the temperature of the sample in the chamber 14 from the moment it is placed in the chamber until the moment it is removed from the chamber for the clinical assay. Sensor 26 is operatively connected to storage chip 28 that stores the collected temperature information from the sensor 26 and then downloads the stored information to a data processing unit for the quality evaluation step. The sensor 26 is triggered or actuated by fluid responsive element 27 positioned at the bottom of the chamber 14.

In a variation of the above embodiment, shown in FIG. 1B, cap 23 is a security cap that is triggered or actuated upon placement of the cap on the top of the tube following sample obtainment. Between Cap 23 and chamber wall 16 is an electronic seal 29 that, if compromised, produces a signal that is recorded in recording element 28, where the signal is subsequently employed in the sample evaluation step, e.g., to determine that the sample was compromised by exposure to air at some point during storage.

As stated above, any convenient sample containment device that provides the above functionality may be employed, where the above representative embodiments have been provided for illustrative purposes only.

### EMBODIMENTS WHERE ADDED QUALITY INDICATORS ARE EMPLOYED

In certain embodiments, as described above, a quality indicator element is added to the sample at the time of sample obtainment, where the quality indicator element is one that that is employed in the quality evaluation step to determine the quality of the sample. In these embodiments, the quality indicator element, made up of one or more individual components, is an element that is added to the sample at a time prior to the quality evaluation step, e.g., at the time of sample obtainment, and is then employed in the quality evaluation step to determine the quality of the sample at the time of clinical assay.

A variety of different types of quality indicator elements may be employed in these embodiments of the subject invention. In one representative embodiment, the quality indicator element is a nucleic acid indicator element, where the indicator element is made up of one or more distinct nucleic acids that are detected in the evaluation step. For example, the nucleic acid quality indicator element may be made up of one or more “canary” nucleic acids that are modulated as the sample into which they are placed degrades. In this embodiment, at the quality evaluation step the “canary” nucleic acids are detected, where the result obtained is employed to determine the quality of the sample. Any convenient “canary” nucleic acid(s) may be employed, where in many embodiments the “canary” nucleic acids are ones that are not nucleic acids found in the sample that is being clinically assayed. Representative “canary” nucleic acids of interest include, but are not limited to: xenogenes, such as plant genes not found in any animal sample and the like. Another nucleic acid quality indicator element of interest is one or more ribonucleic acid molecules, e.g., synthetic RNA(s), where the molecules “age” as the sample ages, such that detection of the RNA at the quality evaluation step provides a measure of the quality of the sample. Any convenient ribonucleic acid molecules may be employed, where in many embodiments of interest the sequence of the RNA molecules is a sequence not found in any of the nucleic acids in the sample being assayed. Examples of this type may be viral or bacterial RNA molecules, or any other of a species different from the sample type and thus easily differentiated. Synthetic RNA molecules may also be RNAs modified from the form in which they are found naturally, e.g. so-called armored RNA, or RNA having different structural elements. Another nucleic acid quality indicator element of interest is a cell that ages in parallel with cells in the sample, where the cell harbors nucleic acids that, at the time of quality evaluation, provide an indication of the quality of the sample. For example, dried yeast cells may be added to the sample upon obtainment. Upon contact with the sample, the dried yeast cells are reconstituted. At the time of sample evaluation, the sample is assayed for the presence, either qualitatively or quantitatively, of one or more yeast nucleic acids, where the results are employed to determine the quality of the sample.

Where the quality indicator element is made up of one or more distinct nucleic acids, the one or more nucleic acids of the quality indicator element may be detected, either qualitatively or quantitatively, in a variety of different ways, as a number of different nucleic acid detection protocols are known in the art. For example, the sample may be electrophoretically separated and the resultant nucleic acid quality indicator elements detected in the resultant separated nucleic acids, e.g., in a blot procedure, such as a Northern or Southern blot, depending on the specific nature of the nucleic acids.

In many embodiments of interest, the nucleic acid quality indicator element is detected using an array of probe nucleic acids, where the array may be the same as or different from the array that is employed in the clinical assay of the subject methods. In these embodiments, the array includes one or more probe nucleic acids for each constituent nucleic acid member of the nucleic acid quality indicator element.

In other embodiments, the quality indicator element is a signal producing system made up of one or more components, where the signal producing system produces a signal that is employed at the quality evaluation step to determine the quality of the sample. Any convenient signal producing system that is affected by a sample quality parameter of interest, e.g., time, temperature, etc., and produces a signal reflective of such may be employed. Signal producing systems finding use as quality indicator elements are systems typically made up of one or more chemical reagents that work together to produce the signal employed in the quality evaluation step, i.e., the quality indicative signal.

Any convenient signal producing system that produces a usable quality indicative signal may be employed in the subject methods. One representative signal producing system is made up of a FET (fluorescence energy transfer) or FRET (fluorescence resonance energy transfer) labeled reagent whose signal changes upon degradation, where the degradation of the agent can be correlated to the degradation of the sample and therefore employed to determine the quality of the sample. For example, one may use a FET labeled reagent, compound or molecule that contains both donor and quencher moieties that are separated from each other by an enzyme substrate for an enzyme that is only present when the sample has degraded and is therefore of unacceptably poor quality. For example, a substrate for an intracellular protease, nuclease, etc., that is only present in the media upon degradation of the sample, e.g., as embodied by disruption of cellular integrity and consequent leakage of intracellular components into the extracellular environment, may be present on a linker between a donor and acceptor on a FRET construct probe. The presence of the enzyme cleaves the reagent at the substrate thereby separating the donor from the quencher. As such, detection of fluorescence can be used as a signal that the sample has been unacceptably degraded, i.e., can be used to determine the quality of sample. Suitable FRET constructs that may be employed in the present invention are known in the art, see e.g., U.S. Pat. No. 5,981,200; the disclosure of which is herein incorporated by reference.

In an alternative embodiment, one may include in the sample an enzyme whose activity can be correlated with the quality of the sample at the time the sample is evaluated for quality. As such, in these embodiments, the activity of the quality indicator enzyme is assayed at the quality evaluation step, where the resultant activity reading is employed to determine the quality of the sample. By introducing enzymes such as β-galactosidase or horse radish peroxidase and their appropriate chromophoric substrates at the time of sample collection or following the initial processing of the sample, one can easily monitor how effective the storage condition has been. If the sample has been properly stored there should only be baseline level of color development during transit and storage. However, if the sample has been abused and enzymatic activity allowed, the substrate will be degraded resulting in the development of a colored solution which can be detected spectraphotometricly.

In yet other embodiments, an enzymatic assay that measures the activity of one or more enzymes intrinsic to the sample may be employed. For example, one could measure or evaluate trypsin activity in a buccal smear biopsy. The enzymes could be secreted enzymes that could be present in the fluid (e.g. blood or serum) that a given biopsy (e.g. tumor) is collected in.)

In yet other embodiments, the quality indicator element is a device or mechanical element that is added to the sample, e.g., at the time of obtainment. The device or mechanical element may be present in a convenient configuration for adding to the sample, e.g., in the shape of a bead or other structure that fits in the sample containment element, where the device may include one or more sensors, as described above.

### COMBINATION EMBODIMENTS

In certain embodiments, a combination of two or more of the above quality evaluation approaches is employed in the subject methods. For example, quality of the sample may be assessed using both a result obtained from a quality indicator element added to the sample at the time of obtainment and an assay for contaminants that are present in the sample. In another representative example, the quality of the sample may be assessed using the above inputs, as well as input regarding one or more physical parameters of the sample, such as temperature during storage, exposure to air/environment, etc, as described above.

Regardless of the particular manner in which the quality of the sample is evaluated, as is apparent from the above discussion, a feature of the subject methods is that sample quality is determined separately or independently from the clinical assay, such that it is not simply derived from the clinical assay results themselves, but is determined using separate quality assay results.

As mentioned above, the quality evaluation step may occur before or at the same time as, the clinical assay, depending on the particular quality evaluation protocol and/or the desirability of having the quality results before running the clinical assay, e.g., as in those embodiments where the clinical assay is performed only if the quality evaluation results meet a predetermined threshold criterion or set of criteria.

In certain embodiments, the overall clinical array based protocol employed is one in which the clinical assay step of the subject protocol is not practiced unless the result of the quality evaluation step, i.e., the sample quality signature, satisfies a predetermined criterion or set of criteria. In these embodiments, the quality evaluation step is performed first and, depending on the result thereof, the clinical assay step is or is not performed.

Programming

Programming for practicing certain embodiments of the subject methods is also provided. For example, algorithms that are capable of directing an array reading device, e.g., an array scanner, to perform a quality evaluation step and/or to perform a clinical assay only if the sample quality meets a predetermined value, are provided. For example, in those embodiments where an array is employed first in the quality evaluation step, the result of the quality evaulation, e.g., in the form of an RF signal, can then be forwarded to the clinical assay system, which will or will not be run on the sample depending on the forwarded result. Viewed another way, the programming of this embodiment at least instructs a reading device to associate or correlate a quality measure of a sample with a sample. The programming then may instruct the reading device to take some further action, e.g., clinically assay the sample, report a result from a clinical assay of a sample, etc., based on whether the quality measure of the sample meets a certain quality threshold.

Programming according to the present invention can be recorded on computer readable media, e.g., any medium that can be read and accessed directly or indirectly by a computer. Such media include, but are not limited to: magnetic tape; optical storage such as CD-ROM and DVD; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above-described methodology. In certain embodiments, the programming is further characterized in that it provides a user interface, where the user interface presents to a user the option of selecting among one or more different, including multiple different, quality criteria, etc.

Utility

The subject invention finds use in clinical array-based assays, particularly where a sample to be assayed is obtained at a first location by a first individual, stored for a period of time and then clinically assayed or tested at a second location by a second individual. As indicated above, the clinical array based assay to which the sample is subjected in the subject methods may be a diagnostic assay, e.g., where the presence of a certain condition, such as a disease condition, is determined; or part a therapeutic regimen, e.g., to monitor the progression of the disease condition.

The subject methods may be used to detect/monitor any condition whose presence and/or state is associated with a defined biopolymeric, e.g., genomic or proteomic, profile, such that a determined biopolymeric profile can be used to determine the presence of state of the condition of interest. A variety of conditions may be detected and/or monitored according to the subject invention. Representative conditions that are amenable to detection and/or monitoring using array-based assays include, but are not limited to: neoplastic disease conditions, cardiovascular disease conditions, pathogenic disease conditions (such as viral disease conditions), neurological, immune function and the like. Additional applications of interest include, but are not limited to: population screening protocols, where the people being monitored are “normal,” e.g., in haploptyping protocols.

In practicing the subject methods, the array-based clinical assay component of the subject methods may be viewed as an analyte detection application, in which the presence of a particular analyte(s) in a given sample is detected at least qualitatively, if not quantitatively. Protocols for carrying out such assays with arrays are well known to those of skill in the art and need not be described in great detail here. Generally, the sample is contacted with an array under conditions sufficient for the analyte(s) (if present) to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label present on the analyte, etc. The presence of the analyte in the sample is then deduced from the detection of binding complexes on the substrate surface.

Specific clinical array-based assay applications of interest include hybridization assays in which a nucleic acid array is employed. In these assays, a clinical sample is first obtained and then prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assay protocols that may be employed in a given clinical array based assay include: simple contact with an array; differential gene expression analysis assays where the sample is compared to a reference; and the like.

Patents and patent applications describing methods of using nucleic acid arrays in various applications, including clinical array diagnostic applications, include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Patents and patent applications describing methods of using proteomic arrays in various applications, including clinical array diagnostic applications, include: U.S. Pat. Nos. 4,591,570; 5,171,695; 5,436,170; 5,486,452; 5,532,128; and 6,197,599; the disclosures of which are herein incorporated by reference; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

In certain embodiments, the subject methods include a step of transmitting data from at least one of the quality evaluation and clinical assay steps, as described above, where the transmitted date may include both the clinical assay results and the quality results, where the data may be processed or not, as described further below. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in practicing the methods of the subject invention, the array will typically be exposed to a clinical sample (for example, a clinical sample that has been fluorescently labeled) and the array then read. Reading of the array may be accomplished by illuminating the array and reading the location and intensity of resulting fluorescence at each feature of the array to detect any binding complexes on the surface of the array. For example, a scanner may be used for this purpose, such as the AGILENT MICROARRAY SCANNER device available from Agilent Technologies, Palo Alto, Calif. Other suitable apparatuses and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Array Readers

Also provided by the subject invention are biopolymer array optical readers or scanners that are programmed as described above, e.g., to perform an independent quality evaluation step and/or to perform a clinical assay, i.e., read an array, only if a sample meets a predetermined threshold criterion. Any biopolymer optical scanner or device may be provided to include the above programming. Representative optical scanners of interest include those described in U.S. Pat. Nos. 5,585,639; 5,760,951; 5,763,870; 6,084,991; 6,222,664; 6,284,465; 6,329,196; 6,371,370 and 6,406,849—the disclosures of which are herein incorporated by reference.

Systems

Also provided by the subject invention are systems for performing the array-based clinical assay protocols described herein. The systems include at least the following components: (a) a biological sample containment device, e.g., such as the sample containers described above; a sample quality assay element for assaying said sample to obtain a quality result, e.g., such as the quality indicator elements that are added to the sample (as described above), (b) elements for use in detecting contaminants in the sample, e.g., a nucleic acid array, etc.; and (c) a clinical assay array for assaying the sample to obtain a clinical assay result. The systems may further include a number of additional components that may find use in a given protocol, e.g., sample preparation reagents, labels, etc., where representative embodiments of such components are described elsewhere.

Kits

Kits for use in analyte clinical assays according to the present invention are also provided. The kits at least include one or more components employed in a clinical sample quality evaluation step, as described above. As such, the kits may include one or more of: reagents for detecting the presence of contaminants in a sample, e.g., an array for detecting nucleic acid contaminants; quality indicator elements and components for detecting the same, e.g., nucleic acid quality indicator elements, signal producing system quality indicator elements, etc.; sample containment elements, e.g., with built in or integrated physical quality measurement components, as described above, where the containment means may be disassembled, e.g., in the form of a tube and separate security cap that is triggered or actuated upon placement on the tube; and the like. The kits may further include one or more additional components necessary for carrying out the array-based clinical assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array-based assays.

The kits may also include a computer readable medium including programming, as discussed above, and instructions. The instructions may include installation or setup directions. The instructions may include directions for use of the invention.

Providing software and instructions as a kit may serve a number of purposes. The combinations may be packaged and purchased as a means of upgrading an existing scanner device. Alternatively, the combination may be provided in connection with a new device for reading arrays, in which the software may be preloaded on the same. In which case, the instructions will serve as a reference manual (or a part thereof) and the computer readable medium as a backup copy to the preloaded utility.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

It is evident from the above discussion that the above-described invention provides a number of advantages to the field of array-based clinical assays. For example, by using the subject invention one can determine the quality of a sample prior to running a clinical assay on the sample, and decide not to run the clinical assay if the sample quality is not acceptable. Such an approach can provide significant resource savings. In addition, by having an independent quality evaluation step for each sample, a lab can track samples that routinely do not have sufficient quality, and take corrective steps for the sample obtainment and storage in such instances. Furthermore, having an independent sample quality assessment can impart additional confidence in test results, thereby increasing the value of such results. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of performing a clinical array-based assay protocol, said method comprising:
assaying a sample in a quality evaluation step to obtain a quality result for said sample; and
optionally clinically assaying said sample by an array-based assay protocol to obtain a clinical assay result for said sample;
wherein said quality result is separate from any clinical assay result obtained in said protocol.

2. The method according to claim 1, wherein said quality evaluation step comprises assaying said sample for at least one quality indicator element.

3. The method according to claim 2, wherein said quality indicator element is a nucleic acid quality indicator element.

4. The method according to claim 3, wherein said quality evaluation step comprises contacting said sample with a nucleic acid array.

5. The method according to claim 4, wherein said nucleic acid array is also employed in said clinical array-based assay protocol.

6. The method according to claim 3, wherein said nucleic acid quality indicator element is intentionally added to said sample during said protocol.

7. The method according to claim 3, wherein said nucleic acid quality indicator element is intentionally added to said sample prior to said protocol.

8. The method according claim 3, wherein said nucleic acid quality indicator element is a nucleic acid contaminant that is not intentionally added to said sample during said protocol.

9. The method according to claim 2, wherein said quality indicator element comprises a signal producing system made up of one or more reagents that produces a sample quality indicative signal.

10. The method according to claim 1, wherein quality evaluation step comprises assessing at least one physical characteristic of said sample.

11. The method according to claim 10, wherein said assessing comprises determining at least one physical condition to which said sample has been subjected during said clinical assay protocol.

12. The method according to claim 1, wherein a clinical assay result is obtained only if said quality result satisfies a predetermined criterion.

13. A method comprising transmitting a clinical assay result and quality result obtained by a method of claim 1, from a first location to a second location.

14. The method according to claim 13, wherein said second location is a remote location.

15. A method comprising receiving a transmitted result of an assay performed according to the method of claim 1.

16. A method of performing a clinical array-based assay protocol, said method comprising:
(a) combining a sample with at least one quality indicator element upon obtainment of said sample; and
(b) assessing said at least one quality indicator element to obtain a quality result of said nucleic acid sample.

17. The method according to claim 16, wherein said sample is stored for a period of time between said combining and assessing steps.

18. The method according to claim 15, wherein said quality indicator element is a nucleic acid quality indicator.

19. The method according to claim 16, wherein said quality indicator element comprises a signal producing system made up of one or more reagents that produces a quality indicative signal.

20. The method according to claim 16, wherein a clinical assay result is obtained during said method only if said quality result satisfies a predetermined criterion.

21. The method according to claim 16, wherein a clinical assay result is obtained regardless of said quality result.

22. A method of performing a clinical array-based assay protocol, said method comprising:
assaying a sample in a quality evaluation step for the presence of at least one contaminant to obtain a quality result for said sample; and
optionally clinically assaying said sample to obtain a clinical assay result for said sample;
wherein said quality result is separate from any clinical assay result obtained in said protocol.

23. The method according to claim 22, wherein said sample is assayed for two or more different contaminants.

24. The method according to claim 22, wherein said contaminant is a nucleic acid.

25. The method according to claim 22, wherein a clinical assay result is obtained during said method only if said quality result satisfies a predetermined criterion.

26. The method according to claim 22, wherein a clinical assay result is obtained regardless of said quality result.

27. A method of performing a clinical array-based assay protocol, said method comprising:
assaying a sample in a quality evaluation step for at one physical characteristic to obtain a quality result for said sample; and
optionally clinically assaying said sample to obtain a clinical assay result for said sample;
wherein said quality result is separate from any clinical assay result obtained in said protocol.

28. The method according to claim 27, wherein said sample is assayed for two or more different physical characteristics.

29. The method according to claim 27, wherein said assaying step includes employing a sample containment device that includes at least one sample physical characteristic sensor.

30. The method according to claim 27, wherein a clinical assay result is obtained during said method only if said quality result satisfies a predetermined criterion.

31. The method according to claim 27, wherein a clinical assay result is obtained regardless of said quality result.

32. A sample containment device, said device comprising:
(a) a container element for holding said sample; and
(b) at least one sample quality indicator element.

33. The device according to claim 32, wherein said at least one quality indicator element is a reagent that is combined with said sample when said sample is placed in said container element.

34. The device according to claim 32, wherein said at least one quality indicator element is a mechanical sensor.

35. A system for conducting an array-based clinical assay, said system comprising:
(a) a biological sample containment device;
(b) a sample quality assay element for assaying said sample to obtain a quality result;
(c) a clinical assay array for assaying said sample to obtain a clinical assay result.

36. A computer-readable medium comprising a program that instructs an array reading device to correlate a quality measure with a sample.

37. The computer readable medium according to claim 36, wherein said program further instructions said reading device to clinically assay said sample or report a result from assaying said sample in response to an input that said quality measure associated with said sample satisfies a threshold quality criterion.

38. A kit for use in an array-based clinical assay, said kit comprising:
(a) a sample quality assay element for assaying said sample to obtain a quality result; and
(b) instructions for use in practicing clinical assay method according to claim 1.

39. The kit according to claim 38, wherein said kit further comprises a sample containment element for holding a sample.
